(19) United States
(12) Patent Application Publication
(10) Pub. No.: US 2026/0306107 A1
(43) Pub. Date: Oct. 8, 2026

(54) TOURNIQUET DEVICES, SYSTEMS AND METHODS FOR USING THE SAME

(71) Applicants: Casey G. Little, Franklin, IN (US); Imrich F. Kuric, SR., North Webster, IN (US)

(72) Inventors: Casey G. Little, Franklin, IN (US); Imrich F. Kuric, SR., North Webster, IN (US)

(21) Appl. No.: 19/697,397

(22) Filed: Jun. 3, 2026

Related U.S. Application Data
(63) Continuation of application No. 17/515,485, filed on Oct. 31, 2021, now abandoned.

(51) Int. Cl. A61B 17/132; A61B 17/00

(52) U.S. Cl. CPC A61B 17/1325; A61B 2017/00115; A61B 2017/00132; A61B 2017/00137; A61B 2017/00199

(57) ABSTRACT

Tourniquet devices and systems for emergency and in-field use are provided that comprise a timing apparatus to facilitate accurate tracking of elapsed time from the initiation of treatment. Methods for using a tourniquet or tourniquet system of the present disclosure are also described.

### PRIORITY

This application is related to, a U.S. continuation patent application of, and claims the priority benefit of U.S. patent application Ser. No. 17/515,485, filed Oct. 31, 2021. The content of the aforementioned application is hereby expressly incorporated by reference in its entirety into this disclosure.

### TECHNICAL FIELD

The present disclosure relates to devices, systems, and methods of using emergency tourniquets that comprise easy to use and accurate means of recording initiation time and tracking treatment time. Further, certain embodiments relate to devices for medical emergencies in one or more applications such as, without limitation, military and pre-hospital emergency applications, and methods of operating said devices.

### BACKGROUND

Generally, a tourniquet is a device for stopping the flow of blood through a vein or artery, typically by compressing a limb or other body portion with a cord or tight bandage. Elimination of distal artery flow is the primary purpose of a tourniquet. Most commercially manufactured tourniquets comprise some form of an encircling cuff that can be applied to a limb proximal to an exsanguination site and sufficiently tightened such that sufficient pressure is achieved to stop arterial and venous blood flow past the cuff (i.e. obliterating the distal pulse).

There are many types of conventional tourniquets. Clinical tourniquets can be simple elastic bands used in a hospital or clinical setting for diagnostic and interventional purposes. Such tourniquets are commonly used in peripheral intravenous access insertion and blood extraction, but typically cannot achieve the requisite pressures to stop arterial bleeding. For example, a clinical tourniquet can be tightened just enough to permit blood to collect in a vein to be accessed, but is typically not suitable in a triage setting.

In contrast, surgical tourniquets are typically used by surgeons or other health care practitioners to facilitate surgical procedures on upper and lower limbs; in use these tourniquets are deployed to establish a bloodless operative field in a limb distal to an encircling cuff by regulating a pressure applied to the limb by the cuff near a pressure sufficient to stop arterial blood flow past the cuff. Such tourniquets often comprise an inflatable pneumatic cuff that can produce enough pressure to impede blood flow, but can easily be decompressed if the pressure needs to be relieved. Often, surgical tourniquets also comprise an automatic pressure regulator and/or the pneumatic mechanism is electrically powered such that the tourniquet itself comprises wires and other electrical components.

Emergency tourniquets are the third type of tourniquets and are typically carried by military medics, paramedics, and rescue personnel. Conventional emergency tourniquets do not comprise electrical componentry, but are instead lightweight, sturdy, and durable bands or straps that can be applied quickly and, often, single-handedly. Examples of emergency non-pneumatic tourniquets are a combat application tourniquet (CAT) and a ratchet tourniquet. A conventional CAT tourniquet consists of a hook-and-loop, closed-loop fastener belt attached to a windlass mechanism, and is among the most widely used tourniquets in the United States Army. The CAT is designed for rapid, one-handed self-application in the field and is applied by manually twisting the belt with the windlass. Similar to the CAT, a traditional ratchet tourniquet forms a loop that must be fed over the injured limb and advanced upward past the injury before tightening high up on the limb. Unlike the plastic and cloth assembly of the CAT, the metal ratchet mechanism of a ratchet tourniquet forms a strong bond that is unlikely to dislodge with even the roughest handling. Pneumatic tourniquets have also been developed for military and emergency use, based on surgical designs considerations. While these tourniquets typically do not comprise electrical componentry and regulators like conventional surgical tourniquets, they do comprise a wide cuff coupled with a windlass and a hand bulb pump. In one such pneumatic tourniquet design, the emergency and military tourniquet (EMT), a hand pump is manually inflated to create pressure and stop bleeding.

When tourniquets are applied, the potential for injury is significant when protocols are not rigorously followed. Preventing arterial blood flow to a limb will result in ischemia. Continuous tourniquet application can, for example, result in permanent nerve injury, gangrene, muscle injury (including contractures, rhabdomyolysis, and compartment syndrome), vascular injury, skin necrosis, clot, significant pain, reperfusion injury, and even amputation of the limb.

There is no consensus on an absolutely safe duration for tourniquet use, however, a range of 1-3 hours has been suggested, with 2 hours largely considered a useful guideline for safe usage during elective surgery. It has also been suggested that the effects of traumatic injury and blood loss (e.g., due to an injury during combat and/or where the patient has suffered a medical trauma) can reduce the ischemic tolerance of the limb in comparison to elective surgery, suggesting that safe tourniquet times may be shorter than expected. especially for patients in shock.

In general, minimizing the tourniquet time is the most effective strategy to minimize the risks of tourniquet-related injury. Along these lines, accurately tracking how long the tourniquet has been applied to a patient is critical information for not only preventing permanent damage to the patient's limb, but also enabling a healthcare provider to quickly and accurately assess the most pressing risk to the patient upon assumption of care. For example, where an emergency tourniquet is applied in a field triage setting, it is extremely important that a receiving healthcare team is provided with an accurate duration of use of the tourniquet so that an accurate risk assessment can be performed in determining the proper, and most urgent, course of treatment. The current standard of care is that an initiation time is handwritten (e.g., with a marker) on the tourniquet when applied. As an emergency tourniquet is often applied in the field at a time of high stress for everyone involved, the likelihood of an accurate and legible time being recorded on the tourniquet and thereafter remaining visible and legible is often low.

Accordingly, devices and methods that can reliably and accurately record the duration of tourniquet application are needed. Ideally, such devices and methods should be cost effective to manufacture, tamper resistant, and simple to use in a field environment.

### SUMMARY

Tourniquets configured for emergency and/or field (non-or pre-hospital) that facilitate the simple and accurate tracking of elapsed treatment time are provided. In certain embodiments, such a tourniquet comprises a flexible band for encircling and applying pressure to a limb of a patient, a fastening element for securing a first portion of the flexible band to a second portion of the flexible band, a tensioning device operative to apply tension to the flexible band, and a timer apparatus. The tensioning device of the tourniquet can comprise any tensioning device now known or hereinafter developed that is appropriate for use with the tourniquet designs described herein. In certain embodiments, the tensioning device comprises at least one of a ratchet, a windlass, a slip buckle, a frictional bias means, a hook-and-loop fastener and a hand bulb pump. In certain embodiments, the tensioning device does not comprise electrical circuitry or otherwise require a pump or actuator. In certain embodiments, the tourniquet is configured for single-handed, self-application.

In certain embodiments, the timer apparatus is affixed to the flexible band (e.g., removably affixed or semi-permanently affixed or securely, albeit removably, affixed). In certain embodiments, the timer apparatus is integrally formed with the flexible band (i.e. integral with the flexible band).

The timer apparatus can comprise a housing, a display coupled with the housing, a power supply, a switch operatively controlled externally to the housing, and a microcontroller responsive to closure of the switch and in electrical communication with the display. In certain embodiments, the housing of the timer apparatus is formed of the following materials and/or configured to be one or more of shock resistant, waterproof, or water resistant. The timer apparatus can be configured such that, once activated, the timer cannot return to the inactive condition without opening the housing (e.g., where the housing is not configured to be opened, by breaking the housing or forcing the housing to open) or exhausting the power supply.

In certain embodiments, the power supply of the timer apparatus comprises one or more batteries.

In certain embodiments, the microcontroller of the apparatus can be mounted on a printed circuit board and in electrical communication with a voltage regulator; however, it will be understood that various acceptable configurations can be employed by one of ordinary skill in the art to achieve the same design outcome.

In operation, closing the switch of the timer apparatus can initiate voltage flow between the power supply and the microcontroller, thereby activating the microcontroller to measure elapsed time starting from activation. In certain embodiments, the switch of the timer apparatus comprises an activating component that is removably attachable to the housing and which closes the switch upon removal. In certain embodiments, when the activating component is attached to the housing, the switch is open and the microcontroller is in an inactive condition. The activating component can comprise a release pin, a release tab, or a similar design that is positioned between the microcontroller and the power supply when attached to the housing. In certain embodiments, the activating component is configured for single use.

Tourniquet systems are also provided. Such a tourniquet system can comprise any of the tourniquets described herein or otherwise conventional tourniquets wherein a timer apparatus of the present disclosure is coupled therewith. For example, certain embodiments of a tourniquet system comprise a tourniquet comprising a flexible band for encircling and applying pressure to a limb of a patient, a fastening element for securing a first portion of the flexible band to a second portion of the flexible band, and a tensioning device operative to apply tension to the flexible band. Additionally such systems comprise a timer apparatus coupled with the tourniquet, wherein the timer apparatus comprises: a housing, a display coupled with the housing, a power supply positioned within the housing, a switch (e.g., single-use switch) operatively controlled externally to the housing, and a microcontroller responsive to closure of the switch, positioned within the housing, and in electrical communication with the display. The switch can comprise an activating component operable to close the switch upon removal and closure of the switch can initiate voltage flow between the power supply and the microcontroller, thereby activating the microcontroller to measure elapsed time starting from activation.

In certain embodiments the display of the timer apparatus comprises a digital display, the power supply comprises one or more batteries, the microcontroller of the timer apparatus is mounted on a printed circuit board and in electrical communication with a voltage regulator, the activating component of the switch comprises a pin or a tab, and the pin or tab is positioned between the microcontroller and the power supply when attached to the housing such that the microcontroller is in an inactive state.

Methods of applying a tourniquet using the devices and systems hereof are also provided. In at least one embodiment, a method of applying a tourniquet comprises: providing a constriction device comprising a flexible band, a fastening element for securing a first portion of the flexible band to a second portion of the flexible band, a tensioning device operative to apply tension to the flexible band, and a timer apparatus coupled with the flexible band, the timer apparatus comprising: a housing, a display coupled with the housing, a power supply, a switch operatively controlled externally to the housing and comprising an activating component that is removably attachable from the housing and operable to close the switch upon removal; manipulating the flexible band around a body part of a patient; coupling the first end of the flexible band with the second end of the flexible band to encircle the body part; adjusting the tensioning device to apply constrictive pressure around the body part sufficient to restrict at least arterial blood flow through the body part; and removing the activating component from the timer apparatus to activate the microcontroller and initiate tracking of elapsed time of treatment. In certain embodiments, the step of manipulating the flexible band and adjusting the tensioning device can be performed single-handedly.

In certain embodiments, the method can further comprise displaying an elapsed tourniquet time on the display, the elapsed tourniquet time associated with the time elapsed since the timer was activated. Removing the activating component from the timer can be performed, for example, immediately following adjusting the tensioning device to apply constrictive pressure around the body part.

### BRIEF DESCRIPTION OF THE DRAWINGS

The disclosed embodiments and other features, advantages, and aspects contained herein, and the matter of attaining them, will become apparent in light of the following detailed description of various exemplary embodiments of the present disclosure. Such detailed description will be better understood when taken in conjunction with the accompanying drawings, wherein:

FIG. 1 shows a partial view of a tourniquet of the present disclosure;

FIG. 2 shows a flexible band of a tourniquet or tourniquet system of the present disclosure;

FIG. 3 shows a top, planar view of a timer apparatus of the present disclosure;

FIG. 4 shows a schematic diagram representative of the components of the timer apparatus shown in FIG. 3; and

FIG. 5 shows a schematic diagram illustrating the voltage flow through the components of the timer apparatus shown in FIG. 3 when in the activated condition, wherein the placement of the activating component 312 when the timer apparatus is in an inactive condition is represented by a dotted line.

While the present disclosure is susceptible to various modifications and alternative forms, exemplary embodiments thereof are shown by way of example in the drawings and are herein described in detail.

### DETAILED DESCRIPTION

For the purposes of promoting an understanding of the principles of the present disclosure, reference will now be made to the embodiments illustrated in the figures and specific language will be used to describe the same. It will nevertheless be understood that no limitation of the scope of this disclosure is thereby intended and that specific details are set forth, such as particular embodiments, procedures, techniques, etc. to provide a thorough understanding of the present disclosure. However, it will be apparent to one skilled in the art that the present invention may be practiced in other embodiments that may depart from these specific details.

The present disclosure provides novel tourniquets and tourniquet systems that comprise easy-to-use tamper-proof and accurate timing components. Such devices allow for the recordation of an accurate time of initiation in manner that is simple to effect at the time of treatment (which is often high stress). In one embodiment, technical features described herein can be used to construct various embodiments of a tourniquet device. In one approach, the tourniquet comprises a timer apparatus affixed to or integral with a tourniquet, such as a one-handed self-applying tourniquet. Straightforward and dependable methods are also provided for conveying an accurate elapsed time of occlusion to downstream healthcare workers (e.g., upon hospital disposition following trauma care) following the application of a tourniquet in a field setting, for example. In ground-based tactical situations and prehospital or emergency settings, tourniquets can be used as life-saving hemorrhage control devices. Indeed, the immediate threat to life with an actively hemorrhaging extremity injury is addressed with the liberal use of tourniquets. However, an improperly used tourniquet can cause loss of the limb, compromised limb salvage, and systemic effects harmful to the patient, all of which may result from excess pressure or prolonged, continuous tourniquet time. The longer the tourniquet is in place, the more tissue destruction occurs and the higher the risk for injury (including, for example, reperfusion injury and kidney failure) and the general rule of thumb is reassessment of a tourniquet should occur as soon as the tactical situation permits, but no more than two hours after initial placement.

Excessive occlusion time can result from prolonged transport/evacuation times, or medical attendants at the next level of care either not being aware a tourniquet was applied or not knowing when the tourniquet was applied. As the length of time a tourniquet is applied is highly relevant to risk assessment for the patient, conventional protocols require the individual or healthcare provider applying the tourniquet to mark an initiation date and time on the tourniquet or the patient's head or limb with a marker. However, due to the nature of trauma care, it is often difficult for the person applying the tourniquet to record the date/time information legibly, if at all. Further, due to the very nature of the circumstances in which tourniquets are required, there is quite often blood and other materials present that can distort, obscure, or wash off the handwritten information. With conventional emergency tourniquets there is no guarantee that when the patient is delivered to a hospital or secondary care setting, the receiving healthcare providers will receive or be able to interpret any date/time information provided and, as such, accurately assess patient risk.

While recordation and tracking of elapsed tourniquet time has largely been addressed in the surgical setting with the pneumatic surgical tourniquet commonly used in most modern operating rooms (which includes a tourniquet microprocessor-based controller that supply and/or regulate pressure and a pneumatic tourniquet cuff applied to the limb), use of these complex devices is typically not available or practical in pre-hospital, emergency, and military settings. For example, instead of a surgical team of multiple individuals applying the tourniquet to a patient within a sterile surgical field, emergency tourniquets are commonly configured for self-application in a pre-hospital setting, and often for single-handed application (e.g., where the afflicted limb is an arm). Further, it is not practical for individuals to carry a pneumatic surgical tourniquet on their person when in the field.

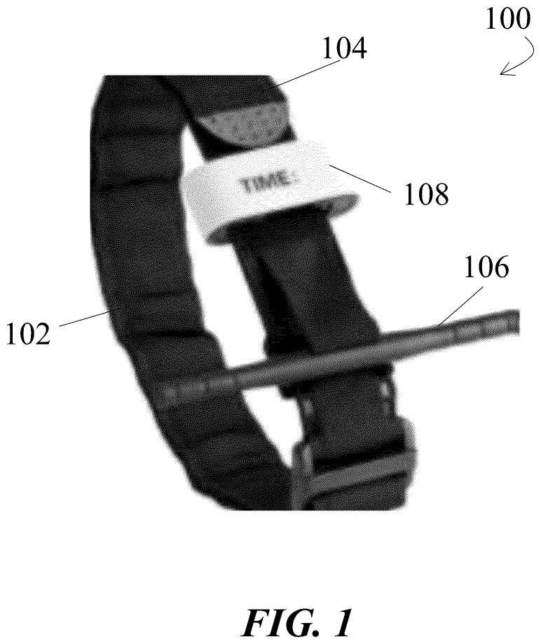

The novel tourniquets of the present disclosure address these issues. Now referring to FIG. 1, a tourniquet 100 of the present disclosure is shown. The tourniquet 100 comprises a flexible band 102 comprising a fastening element 104, a tensioning device 106, and a timer apparatus 108.

The flexible band 102 is configured for encircling and applying pressure to a limb of a patient and can be opened ended or a closed-loop band. In certain embodiments, the flexible band 102 can be a strap, a belt, an elastic band, or a cuff (e.g., an inflatable cuff having a hollow interior). The flexible band 102 can be any diameter in width and length, as needed. In certain embodiments, the flexible band is at least about 1.0 inches in width, between about 1.0 inches and 2.0 inches, and/or between about 1.5 inches and 2.0 inches (e.g., 1.5 inches, 1.6 inches, 1.7 inches, 1.8 inches, 1.9 inches, 2.0 inches, 2.5 inches, etc.). In some instances, wider flexible bands 102 can be better tolerated by a patient and are less likely than narrower bands (e.g., less than 1 inch wide) to cause damage to underlying structures such as nerves and soft tissue present within the underlying limb.

The flexible band 102 can be stretchable so that it can be wrapped around the limb and thereby compress the limb in use. In certain embodiments, the flexible band 102 has a spring constant such that it can be stretched enough to exert sufficient pressure on a limb to exceed the blood pressure causing bleeding. The spring constant can be in the range of, for example, about 1 N/m to about 300 N/m or as is otherwise known in the medical arts. A tourniquet 100 used to control arterial bleeding should be able to exert enough pressure to overcome the arterial systolic pressure, for example, 80 mmHg, 100 mmHg, 120 mmHg, 150 mmHG, and/or 200 mmHg.

The flexible band 102 can be made from a sheet or strip of material suitable for tourniquet applications, including, without limitation, a synthetic polymer nylon, grow ribbon, elastic, polyester, hook and loop fastener material or any combination thereof. Non-limiting examples of hook-and loop-fastener material is Velcro®, or self-engaging fastener material which is material with hook and loop woven on the same surface such as snag-free fasteners known as Omni-tape®. The flexible band 102 can also be made from a thermoplastic body material, for example, comprising or including polypropylene. The flexible band 102 can be formed as a single piece or section of such material or as any combination of sections or straps of the material, as needed.

In certain embodiments, the flexible band 102 is made from a webbing material, such as that typically used in seat belt webbing. The webbing can be polyester webbing or a webbing made from, or including, a synthetic polymer (e.g., nylon).

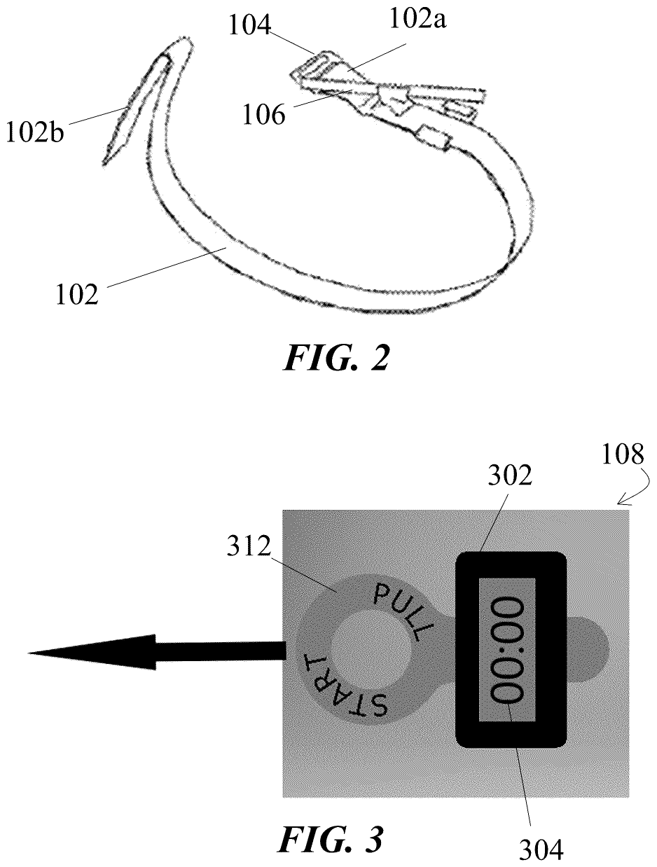

As shown in FIG. 2, in certain embodiments, the flexible band 102 can have a first end 102a and a second end 102b opposite the first end 102a, where the first and second ends 102a, 102b are configured to engage with each other via the fastening element 104. In the embodiment shown in FIG. 2, the flexible band 102 is generally elongated and planar in configuration and has a hollow longitudinal interior. In other example, other shaped configurations can be adopted.

The fastening element 104 is either integral with or coupled with the flexible band 102 and is for securing the flexible band 102 around the underlying limb to be treated. For example, the fastening element 104 can be a fastener configured to releasably interconnect the first and second ends 102a, 102b of the flexible band 102 together (see, e.g., FIG. 2).

The fastening element 104 can be any type of fastener known or future developed that is capable of releasably closing the flexible band 102 in a loop around the limb to be treated. Numerous configurations and designs of fastening elements 104 are known in the tourniquet arts and any of which that are suitable for emergency, field, and/or pre-hospital application can be applied in the present context. For example, the fastening element 104 can comprise a pair of mating members configured to releasably attach to one another, such as, for example, a hook-and-loop fastener. The hook-and-loop fastener can be made of a polyester, polypropylene, aramid, stainless steel, or any other suitable material or combination of the foregoing to suit desired performance characteristics. In the embodiment shown in FIG. 1, at least one hook strip is attached on an inner surface of a first portion of the band 102 and the mating loop strip is attached along an outer surface of a second portion of the band 102 such that the fastener 104 releasably engages itself when in physical contact and holds the flexible band 102 in a loop configuration.

In other embodiments, the fastening element 104 can be made from Velcro® or a snag-free, self-engaging fastener material known as Omni-tape®. In other embodiments, the fastening element 104 can comprise snapping buttons or other hardware such as a buckle. At least a portion of the fastening element 104 can be slidable along the flexible band 102 (e.g., a slider component), and/or some or all of the fastening element 104 can be fixedly attached to the flexible band 102.

The tourniquet 100 further comprises a tensioning device 106 operative to apply tension to the flexible band 102. In use, when the flexible band 102 is positioned around a limb, the tensioning device 106 can adjust the flexible band 102 loop circumference and thereby the tension of the band 102 around the underlying limb. In this manner, the tensioning device 106 can apply constrictive pressure around the body part sufficient to restrict arterial blood flow through the body part.

The tensioning device 106 can be any tourniquet tensioning device now known or hereinafter developed including, without limitation, a windlass mechanism (e.g., a retractable windlass bar or other mechanism), a ratchet, a slip buckle, a frictional bias means, and/or a pneumatic hand pump.

In certain embodiments, the fastening element 104 and the tensioning device 106 can be a single component (e.g., a ratchet or a slip buckle) operable to both secure the flexible band 102 around the targeted area (e.g., limb) of the patient and also apply tension to the flexible band 102 such that circumferential pressure is applied to the limb. It will be appreciated that, in certain embodiments, the flexible band 102, the fastening element 104, and the tensioning device 106 can comprise any conventional tourniquet configuration that is suitable for emergency use and/or use in a pre-hospital setting. In at least one embodiment, the tourniquet configuration suitable for emergency use and/or in a pre-hospital setting has a tensioning device 106 that does not comprise electrical circuitry.

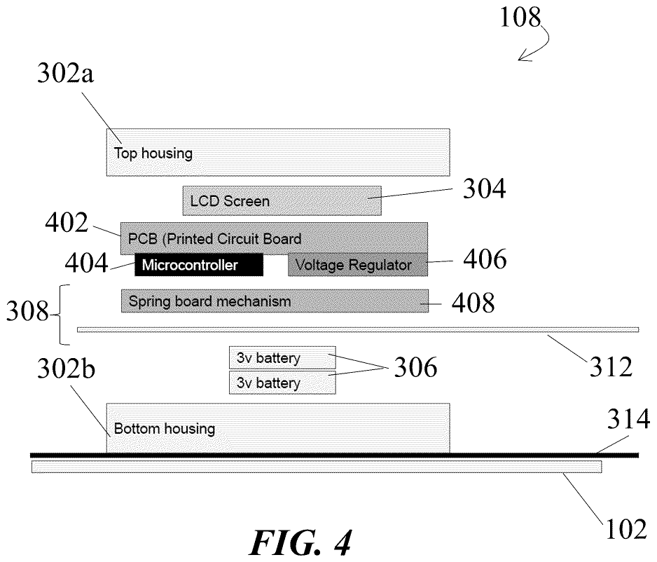

The tourniquet 100 further comprises a timer apparatus 108 coupled with the flexible band 102. As shown in FIGS. 3 and 4, the timer apparatus 108 comprises a housing 302, a display 304 coupled with or otherwise visible by a user through the housing 302, a power supply 306, a switch 308, and a microcontroller 404 responsive to closure of the switch 308 (e.g., to measure/count and record elapsed time). As noted above, the switch 308 of the timer apparatus 108 can comprise an activating component 312 that is removably attachable to the housing 302 and which closes the switch 308 upon removal.

The housing 302 generally encases at least the electrical components of the timer apparatus 108, can be formed of any suitable material(s), and can be a single integral component or two or more panels or portions coupled together (via a hinge, a fixed joint, a combination thereof, or via another connection). In certain embodiments, the housing 302 comprises a plastic case, a silicon case, a metal case, or a combination of any of the foregoing. In certain embodiments, the housing 302 can be made of a waterproof or water-resistant material, a hypoallergenic material, a flame-resistant material, a shock resistant material, or a combination of one or more of the foregoing.

In at least one embodiment, the housing 302 comprises a plastic case with a silicon cover that provides protection against impact. In certain embodiments, the housing 302 comprises acrylonitrile butadiene styrene (ABS). In certain embodiments, the housing 302 is flame-resistant (e.g., having a flammability rating of UL94V-0, UL9HB, and/or MVSS 302).

In certain embodiments, a bottom portion 302b of the housing 302 comprises an attachment means 314 for coupling the timer apparatus 108 to the flexible band 102 of the tourniquet 100. In certain embodiments, the attachment means 314 comprises a hoop-and-loop fastening material (e.g., Velcro®), wherein a compatible material is present on the flexible band 102 at a targeted point of attachment. In this manner, the timer apparatus 108 can be removably coupled with the flexible band 102 simply by aligning the fastening materials and applying pressure. The attachment means 314 can also comprise an adhesive (permanent or otherwise) applied to the bottom portion 302b of the housing 302 such that the housing 302 can be adhered to the flexible band 102 or another portion of the tourniquet 100.

The display 304 of the timer apparatus 108 can be any display now known or hereinafter developed that can visually display a representation of the counter of the microcontroller 404. For example, the display 304 can be an analog or digital display similar to a stopwatch or watch face. For example, the display 304 can comprise a digital screen that displays four digits, representative of “Hr Hr: Min Min” such as, without limitation, a 4-element display that can count from 00:00 to 59:59. In certain embodiments, the display 304 comprises a light-emitting diode (LED) display. In certain embodiments, the display 304 comprises a liquid-crystal display (LCD).

The display 304 can be integral with a top portion 302a of the housing 302 and/or some or all of the top portion 302a of the housing 302 can be transparent such that, when the display 304 is encased within the housing 302, the display 304 remains visible through the transparent portion of the housing 302 such that the timer/counter can be easily visualized. In certain embodiments, at least a portion of the display 304 is coupled with or affixed to the exterior of the housing 302 (e.g., the electrical components of the display 304 can be encased within the housing, with the screen/display portion affixed to and/or integral with an exterior of the top portion 302a of the housing 302). The display 304 can be sized and positioned to facilitate ease of viewing and any size dimensions of the display can be utilized as desired provided the timer apparatus 108 can be suitably affixed to the flexible belt 102 of the tourniquet 100. In certain embodiments, the display 304 can be about 1 inch wide and about 0.5 inches tall. In other embodiments, the display 304 about 2 inches wide and about 1 inch tall.

The timer apparatus 108 further comprises a power supply 306, which powers a microcontroller 404 that is electrical communication with the display 304 when the switch 308 is closed. The power supply 306 can be any suitable power source such as, for example, one or more standard batteries (e.g., a coin cell battery, a button cell battery, a 1.5V or 3V battery, a 5V battery, a 9V battery, a 12V battery, etc.). It will be appreciated that the number and voltage of batteries required and/or desirable can depend on the working voltage of the circuit utilized in the timer apparatus 108.

The microcontroller 404 can be any type of microcontroller capable of performing a time-count in minutes and hours upwards from 00:00 (out of initial) to 59:59 when activated for measuring elapsed time. In certain embodiments, when activated, the microcontroller 404 does not stop, but instead repeats the counting after every full 60-hour cycle unless moved to an inactive condition as described below. In certain embodiments, the microcontroller 404 is an 8-bit microcontroller, a 16-bit microcontroller, or any other size/type of microcontroller as required. In at least one embodiment, the microcontroller 404 is an 8-bit 8051 microcontroller (e.g., a MCS-51 microcontroller).

FIG. 4 shows a schematic of one example of the internal circuitry of the timer apparatus 108. In certain embodiments, the microcontroller 404 is mounted on a circuit board 402 (e.g., a printed circuit board). Certain embodiments can also comprise a voltage regulator 406 and a springboard mechanism 408 that are in electrical communication with the microprocessor 404. The timer apparatus 108 can additionally include one or more transistors, capacitors and/or resistors (not shown) as is known in the art and may be desirable. Further, the microcontroller 404 can be coupled with any additional electrical circuitry or components now known or hereinafter developed provided the microcontroller 404 can be used to measure elapsed time when activated (i.e. in the active condition), encased within the housing 302, and used in connection with the power supply 306 and switch 308 mechanisms described herein.

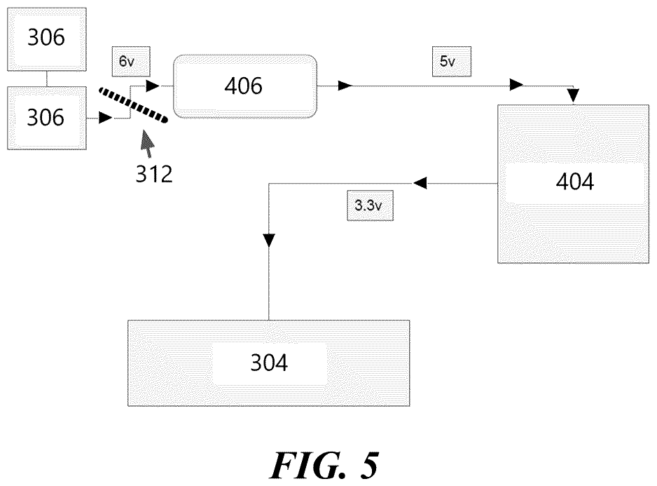

In certain embodiments, the power supply 306 is connected in series with the switch 308 and the switch 308 is operatively controlled externally of the housing (e.g., via the activating component 312). When the switch 308 is open, the microcontroller 404 of the timer apparatus 108 is in an inactive condition; however, closure of the switch 308 initiates voltage flow between the power supply 306 and the microcontroller 404 and activates the measurement of elapsed time from the initial value (i.e. the active condition). FIG. 5 shows a schematic diagram indicative of voltage flow between the various components of the timer apparatus 108.

In embodiments where the switch 308 comprises an activating component 312, in the inactive condition, the activating component 312 is seated between the microcontroller 404 and the power supply 306, which either keeps the power supply 306 disconnected from the rest of the circuit (i.e. the microcontroller 404) or in which the circuit is consuming the lowest quiescent current. The activating component 312 can be, for example, a pull pin or pull tab formed of nonconductive material that is slidable in an axial direction out of its seating. In certain embodiments, the microcontroller 404 is normally in the “inactive” condition.

To initiate the counting functionality of the microcontroller 404 (for example, for measurement of elapsed time), the switch 308 is closed. For example, when the activating component 312 is removed (i.e. slidably pulled from its seating), the switch 308 closes and the power supply 306 is connected to the microcontroller 404/display 304 such that the microcontroller 404 initiates (see, e.g., FIG. 5). Notably, in certain embodiments, once deployed, the timer remains in the “active” condition (i.e. measuring elapsed time) until either the power supply 306 is exhausted or the housing 302 is disassembled and the switch 308 is manually opened; in other words, the switch 308 is not reversible and/or configured for a single use (inactive condition to active condition). In this manner, the timer apparatus 108 is not vulnerable to being erroneously stopped and a user can assure an accurate elapsed time measurement is obtained and visible through the display 304.

In addition to the tourniquet 100 described above, tourniquet systems are also provided. The tourniquet system may comprise any tourniquet now known or hereinafter developed (e.g., an emergency tourniquet comprising a tensioning device that does not comprise electrical circuitry) having a timer apparatus 108 (previously described) affixed thereto. In certain embodiments, the timer apparatus 108 is configured for retrofit application to the band or cuff of the tourniquet. Alternatively, at least a portion of the housing 302 of the timer apparatus 108 can be integrally formed with a portion of the tourniquet (e.g., the band or cuff).

Tourniquet systems of the present disclosure can additionally comprise any tourniquet now known or hereinafter developed, a timer apparatus 108, and an attachment means (not shown). As previously noted, the timer apparatus 108 is formed as previously described. The attachment means can be any attachment means capable of securely affixing the timer apparatus 108 to a portion of the tourniquet and, in certain embodiments, comprises a hook-and-loop fastener or an adhesive. In some embodiments, the attachment means can comprise a first portion for application to the tourniquet and a second portion either already affixed to the bottom portion 302b of the housing 302 of the timer apparatus 108 or configured for application to the bottom portion 302b of the housing 302 of the timer apparatus 108 (e.g., a first Velcro® patch or strip and a second Velcro® patch or strip compatible with the first Velcro® patch or strip).

Methods for using the tourniquets or tourniquet systems described herein are also provided. In certain embodiments, a method of using a tourniquet comprises manipulating the flexible band 102 of a tourniquet 100 (or a conventional tourniquet) around a body party of a patient; coupling the first end 102a of the flexible band 102 with the second end 102b of the flexible band 102 to encircle the body part; adjusting the tensioning device 106 to apply constrictive pressure around the body part sufficient to restrict at least arterial blood flow through the body part; and removing the activating component 312 from the timer apparatus 108 to activate the microprocessor 404 and, thus, initiate tracking of elapsed time of treatment (i.e. from initiation time). In certain embodiments, removing the activating component 312 from the timer apparatus 108 is performed immediately following adjusting the tensioning device 106 of the tourniquet to apply constrictive pressure around the body part. In certain embodiments, manipulating the flexible band 102 and adjusting the tensioning device 106 are performed single-handed.

In certain embodiments, removing the activating component 312 from the timer apparatus 108 is irreversible. In other words, when the switch 308 is closed and the microprocessor 404 moves from the inactive (resting) condition to the active condition, the timer apparatus 108 design does not support reinsertion of the activating component 312 to re-open the switch 308 such that the microprocessor 404 is back in the inactive condition and ceases counting. Instead, the only way to stop the counting functionality of the microprocessor 404 once initiated is to either exhaust the power supply 306 or manually open the switch 308 via direct access (i.e. opening the housing 302 and opening the switch 308 manually). This design feature ensures that the timing apparatus 108 cannot be moved to the inactive condition erroneously once initiated (e.g., if bumped or jostled during transport of the patient or during administration of an additional treatment to the patient).

The present disclosure relates to devices, systems, and methods of using emergency tourniquets that comprise easy to use and accurate means of recording initiation time and tracking treatment time. Further, certain embodiments relate to devices for medical emergencies in one or more applications such as, without limitation, military and pre-hospital emergency applications, and methods of operating said devices.

As used herein and in the appended claims, the singular forms “a,” “and,” and “the” include plural referents unless the context clearly dictates otherwise. Thus, for example, reference to “a battery” includes a plurality of such batteries. The term “about,” when referring to a number or a numerical range, means that the number or numerical range referred to is an approximation within experimental variability (or within statistical experimental error), and thus the number or numerical range may vary between 1% and 15% of the stated number or numerical range. The term “comprising” (and related terms such as “comprise” or “comprises” or “having” or “including”) is not intended to exclude an embodiment of any compound, composition, method, process, or the like that may “consist of” or “consist essentially of” the described features. The invention illustratively described herein may be suitably practiced in the absence of any element(s) or limitation(s), which is/are not specifically disclosed herein.

A “patient” as the terms are used herein is a mammal. While preferably a human, the terms can also refer to a non-human mammal, such as a mouse, cat, dog, monkey, horse, cattle, goat, or sheep, and is inclusive of male, female, adults, and children.

The term “treatment” as used herein includes curative and/or prophylactic treatment. More particularly, curative treatment refers to any of the alleviation, amelioration and/or elimination, reduction and/or stabilization (e.g., failure to progress to more advanced stages) of a symptom, as well as delay in progression of a symptom of a particular disorder. Prophylactic treatment refers to any of the following: halting the onset, reducing the risk of development, reducing the incidence, delaying the onset, reducing the development, and increasing the time to onset of symptoms of a particular disorder.

Unless defined otherwise, all technical and scientific terms used herein have the same meaning as commonly understood by one of skill in the relevant arts. The terms and expressions, which have been employed, are used as terms of description and not of limitation. In this regard, where certain terms are defined, described, or discussed in the “Detailed Description,” all such definitions, descriptions, and discussions are intended to be attributed to such terms. There also is no intention in the use of such terms and expressions of excluding any equivalents of the features shown and described or portions thereof. Although any methods and materials similar to or equivalent to those described herein can be used in the practice or testing of the subject of the present application, the preferred methods and materials are described herein.

While various embodiments of devices, systems, and the methods hereof have been described in considerable detail, the embodiments are merely offered by way of non-limiting examples. Many variations and modifications of the embodiments described herein will be apparent to one of ordinary skill in the art in light of the disclosure. It will therefore be understood by those skilled in the art that various changes and modifications may be made, and equivalents may be substituted for elements thereof, without departing from the scope of the disclosure. Indeed, this disclosure is not intended to be exhaustive or too limiting. The scope of the disclosure is to be defined by the appended claims, and by their equivalents.

Further, in describing representative embodiments, the disclosure may have presented a method and/or process as a particular sequence of steps. However, to the extent that the method or process does not rely on the particular order of steps set forth herein, the method or process should not be limited to the particular sequence of steps described. As one of ordinary skill in the art would appreciate, other sequences of steps may be possible. Therefore, the particular order of the steps disclosed herein should not be construed as limitations on the claims. In addition, the claims directed to a method and/or process should not be limited to the performance of their steps in the order written, and one skilled in the art can readily appreciate that the sequences may be varied and still remain within the spirit and scope of the present disclosure.

It is therefore intended that this description and the appended claims will encompass, all modifications and changes apparent to those of ordinary skill in the art based on this disclosure.

1. A tourniquet comprising:
a flexible band for encircling and applying pressure to a limb of a patient;
a fastening element for securing a first portion of the flexible band to a second portion of the flexible band;
a tensioning device operative to apply tension to the flexible band; and
a timer apparatus comprising a housing, a display coupled with the housing, a power supply, a switch operatively controlled externally to the housing, and a microcontroller responsive to closure of the switch and in electrical communication with the display;
wherein the timer apparatus is affixed to the flexible band and closing the switch initiates voltage flow between the power supply and the microcontroller, thereby activating the microcontroller to measure elapsed time starting from activation.

2. The tourniquet of claim 1, wherein:
the tensioning device is further operative to apply constrictive pressure around the limb of the patient sufficient to restrict at least arterial blood flow through the limb;
the switch of the timer apparatus comprises an activating component that is removably attached to the housing and, when attached to the housing, the switch is open and the microcontroller is in an inactive condition such that the microcontroller does not receive power from the power supply;
removal of the activating component closes the switch, which initiates voltage flow between the power supply and the microcontroller, thereby activating the microcontroller to measure elapsed time starting from activation, and the activation of the microcontroller to measure elapsed time is not reversible so that the measuring of elapsed time is not vulnerable to being erroneously stopped, and
the activating component remains removably attached to the housing after operation of the tensioning device to apply constrictive pressure around the limb of the patient sufficient to restrict at least arterial blood flow through the limb.

3. The tourniquet of claim 2, wherein the activating component is configured for single use.

4. The tourniquet of claim 3, wherein the activating component comprises a release pin or tab that is positioned between the microcontroller and the power supply when attached to the housing.

5. The tourniquet of claim 1, wherein the power supply comprises one or more batteries.

6. The tourniquet of claim 1, wherein the microcontroller is mounted on a printed circuit board and in electrical communication with a voltage regulator.

7. The tourniquet of claim 1, wherein the housing of the timer apparatus is one or more of shock resistant, waterproof or water resistant.

8. The tourniquet of claim 1, wherein at least a portion of the timer apparatus is integral with the flexible band.

9. The tourniquet of claim 1, wherein the timer apparatus is removably affixed to the flexible band.

10. The tourniquet of claim 1, wherein the tensioning device:
comprises at least one of a ratchet, a windlass, a slip buckle, a frictional bias means, a hook-and-loop fastener, or a hand bulb pump; and/or
does not comprise electrical circuitry.

11. The tourniquet of claim 1, wherein once activated, the timer apparatus cannot return to the inactive condition without opening the housing or exhausting the power supply.

12. The tourniquet of claim 1 configured for single-handed, self-application.

13. A tourniquet system comprising:
a tourniquet comprising:
a flexible band for encircling and applying pressure to a limb of a patient,
a fastening element for securing a first portion of the flexible band to a second portion of the flexible band, and
a tensioning device operative to apply tension to the flexible band and apply constrictive pressure around the limb of the patient sufficient to restrict at least arterial blood flow through the limb; and
a timer apparatus coupled with the tourniquet, the timer apparatus comprising:
a housing,
a display coupled with the housing,
a power supply positioned within the housing,
a single-use switch operatively controlled externally to the housing, the switch comprising an activating component operable to close the switch upon removal,
a microcontroller responsive to closure of the switch, positioned within the housing, and in electrical communication with the display such that closing the switch initiates voltage flow between the power supply and the microcontroller, thereby activating the microcontroller to measure elapsed time starting from activation, and the activation of the microcontroller to measure elapsed time is not reversible so that the measuring of elapsed time is not vulnerable to being erroneously stopped, and
wherein when the activating component is attached to the housing, the switch is open and voltage does not flow between the power supply and the microcontroller,
wherein the activating component is removable from the timer apparatus after operation of the tensioning device to apply constrictive pressure around the limb of the patient sufficient to restrict at least arterial blood flow through the limb.

14. The tourniquet system of claim 13, wherein:
the display comprises a digital display;
the power supply comprises one or more batteries;
the microcontroller of the timer apparatus is mounted on a printed circuit board and in electrical communication with a voltage regulator;
the activating component of the switch comprises a pin or a tab; and
the pin or tab is positioned between the microcontroller and the power supply when attached to the housing such that the microcontroller is in an inactive state.

15. A method of applying a tourniquet comprising:
providing a constriction device comprising a flexible band, a fastening element for securing a first portion of the flexible band to a second portion of the flexible band, a tensioning device operative to apply tension to the flexible band, and a timer apparatus coupled with the flexible band, the timer apparatus comprising:
a housing,
a display coupled with the housing,
a power supply,
a switch operatively controlled externally to the housing and comprising an activating component that is removably attachable from the housing and operable to close the switch upon removal thereby providing power to the timer apparatus and activating tracking of elapsed time, and the activation of the tracking of elapsed time is not reversible so that the tracking of elapsed time is not vulnerable to being erroneously stopped, and wherein when the activating component is attached, the switch is open preventing power to the timer apparatus;
manipulating the flexible band around a body part of a patient;
coupling a first end of the flexible band with a second end of the flexible band to encircle the body part;
adjusting the tensioning device to apply constrictive pressure around the body part sufficient to restrict at least arterial blood flow through the body part; and
removing the activating component from the timer apparatus to activate the timer apparatus and initiate tracking of elapsed time of treatment after the step of adjusting the tensioning device to apply constrictive pressure around the body part.

16. The method of claim 15, further comprising displaying an elapsed tourniquet time on the display, the elapsed tourniquet time associated with the time elapsed since the timer apparatus was activated.

17. The method of claim 15, wherein removing the activating component from the timer apparatus is performed immediately following adjusting the tensioning device to apply constrictive pressure around the body part.

18. The method of claim 15, wherein manipulating the flexible band and adjusting the tensioning device are performed single-handed.

19. The tourniquet of claim 1, wherein the housing comprises an attachment means for coupling the timer apparatus to the flexible band at a targeted point of attachment.

20. The tourniquet of claim 4, wherein a portion of the activating component is within the housing and the portion of the activating component within the housing is narrower than a portion of the activating component external to the housing.
